(19) United States
(12) Patent Application Publication
(10) Pub. No.: US 2013/0096424 A1
(43) Pub. Date: Apr. 18, 2013

(54) SYSTEM AND METHOD FOR REAL-TIME ENDOSCOPE CALIBRATION

(71) Applicants: Sheng Xu, Rockville, MD (US); Douglas A. Stanton, Ossining, NY (US)

(72) Inventors: Sheng Xu, Rockville, MD (US); Douglas A. Stanton, Ossining, NY (US)

(73) Assignee: KONINKLIJKE PHILIPS ELECTRONICS N.V., EINDHOVEN (NL)

(21) Appl. No.: 13/702,686

(22) PCT Filed: May 26, 2011
(86) PCT No.: PCT/IB11/52307

§ 371 Date: Dec. 7, 2012

Related U.S. Application Data
(60) Provisional application No. 61/357,122, filed on Jun. 22, 2010.

(51) Int. Cl. A61B 1/018; A61B 1/05; A61B 5/00; A61B 5/06

(52) U.S. Cl. CPC A61B 1/018; A61B 5/065; A61B 1/05; A61B 5/6847
USPC 600/424

(57) ABSTRACT

A sensor tracking device, system and method include a sensor (118) configured on a wire (110) and adapted to fit in a working channel (116) of an endoscope (100). An image identifiable feature (120) is formed on a distal end portion of the sensor which is identifiable when the sensor is extended from the endoscope. An image of the image identifiable feature is collected by the endoscope and permits a determination of a pose of the endoscope.

This disclosure relates to endoscope systems and more particularly to a system and method for endoscope calibration during a medical procedure.

Lung cancer is the leading cause of cancer death in the world. A bronchoscopic biopsy of central-chest lymph nodes is an important step for lung-cancer staging. Before bronchoscopy, a physician needs to visually assess a patient's three-dimensional (3D) computed tomography (CT) chest scan to identify suspect lymph-node sites. During the bronchoscopy, the physician guides a bronchoscope to each desired lymph-node site. Unfortunately, the physician has no link between the 3D CT image data and the live video stream provided during the bronchoscopy. The physician essentially performs the biopsy without real-time visual feedback, which adds difficulty to the procedure.

The development of a virtual bronchoscopy (VB) has led to interest in introducing CT-based computer-graphics techniques into procedures, such as, lung-cancer staging. In VB, interior (endoluminal) renderings of airways can be generated along paths following the airway central axes and lead to an online simulation of live video bronchoscopy. In VB, interior views of organs are computer-generated from radiologic images. This is similar to the situation where real bronchoscopy (RB) views of organs are presented during the procedure.

In accordance with the present principles, VB has made it possible to use computer-based image guidance to assist a physician in performing TransBronchial Needle Aspiration (TBNA) and other procedures. By registering RB and VB, the physician can locate the bronchoscope in the CT dataset. One approach at registering RB and VB is to use electromagnetic (EM) tracking. A 6 degrees-of-freedom EM sensor can be attached to a distal end of the bronchoscope close to a camera. A fixed transformation between a camera coordinate system and a sensor's local coordinate system can be determined by a one-time calibration procedure. The RB/VB fusion can be obtained after registering EM to CT.

In accordance with the present principles, endoscope calibration is provided. In one embodiment, bronchoscope calibration is needed for image guidance in bronchoscopy using electromagnetic tracking. Other procedures (e.g., ultrasound calibration, etc.) and scopes (e.g., colonoscope, etc.) are also contemplated. The transformation between the bronchoscope's camera and the tracking sensor needs to be determined to register a bronchoscopic image to a preoperative CT image. However, it is problematic to attach a tracking sensor to the outside of the bronchoscope because it may complicate the sterilization procedure. On the other hand, the tracking sensor cannot permanently occupy a working channel of the bronchoscope because a standard bronchoscope only has one working channel that is typically used for passing surgical devices. In accordance with one embodiment, a tracking sensor is marked with image identifiable features, allowing a transformation between a bronchoscope's camera and a sensor to be determined in real-time.

A sensor tracking device, system and method include a sensor configured on a wire or cable and adapted to fit in a working channel of an endoscope. An image identifiable feature is formed on a distal end portion of the sensor which is identifiable when the sensor is extended from the endoscope. An image of the image identifiable feature is collected by the endoscope and permits a determination of a pose of the endoscope.
A system for tracking an endoscope includes an endoscope having a working channel, a spatial tracking system and a distally disposed imaging device. A sensor is configured on a wire and adapted to fit in the working channel. At least one image identifiable feature is formed on a distal end portion of the sensor which is identifiable when the sensor is extended from the endoscope. A transformation module is configured to compute a pose of the endoscope by employing a position of an image of the at least one image identifiable feature collected by the imaging device and a position of the spatial tracking system.
A method for tracking an endoscope includes calibrating a transformation between a distally disposed imaging device of an endoscope and a sensor having at least one image identifiable feature formed on a distal end portion of the sensor which is identifiable when the sensor is extended from the endoscope. The endoscope is tracked by passing the sensor through a working channel of the endoscope until the at least one image identifiable feature is imaged and computing a current pose of the endoscope using an image of the at least one image identifiable feature and the transformation.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

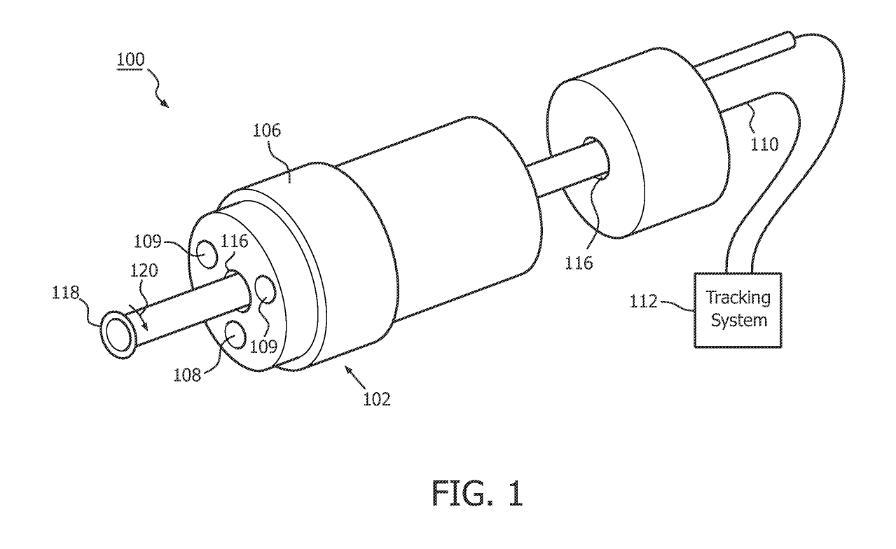

FIG. 1 is a perspective view of an endoscope having a working channel suitable for use in accordance with the present principles;

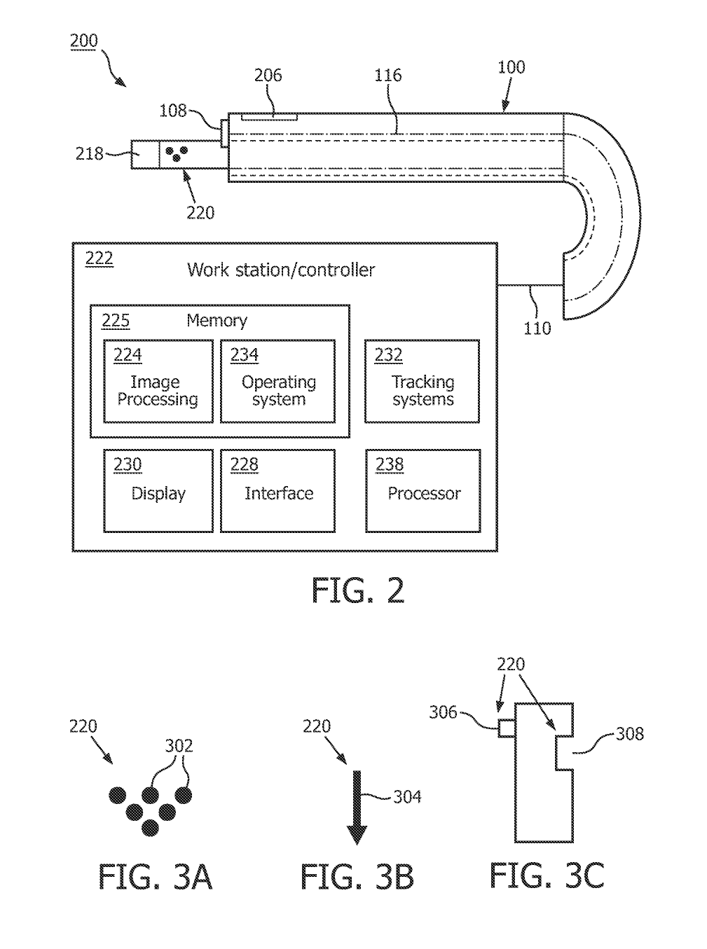

FIG. 2 is a block/flow diagram showing a system for tracking an endoscope in accordance with one embodiment;

FIG. 3A-3C show illustrative examples of an image identifiable feature for use in registering an endoscope with a tracking system;

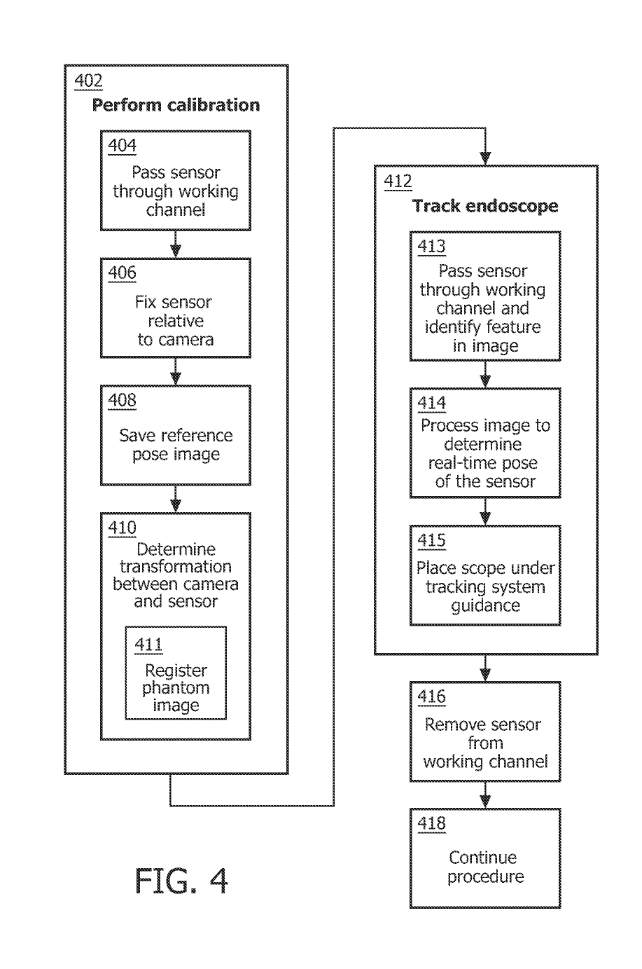

FIG. 4 is a block/flow diagram showing a system/method for registering an endoscope in accordance with the present principles; and

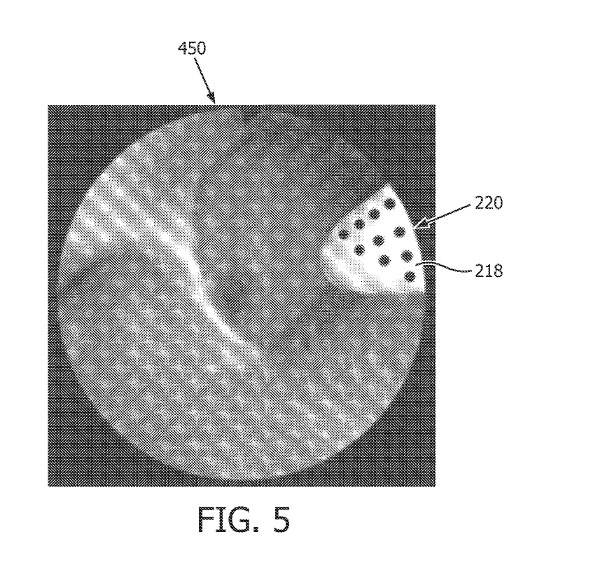

FIG. 5 is an image showing a sensor having an identifiable image feature during a medical procedure.

The present disclosure describes an apparatus, system and method to calibrate an endoscope by registering an endoscopic image to a preoperative image (e.g., a CT image) using a transformation of coordinates between an endoscope camera and a tracking sensor. The tracking sensor is marked with image-identifiable features. In a bronchoscope embodiment, a six degree of freedom (6 DOF) electromagnetic (EM) sensor may be passed in a one-time initial calibration procedure through a working channel of the bronchoscope until the features of the EM sensor can be identified in the bronchoscopic image. When the bronchoscope has to be tracked, the EM sensor is passed through the working channel of the bronchoscope until the features of the EM sensor can be identified in the bronchoscopic image. The bronchoscopic image is then processed to determine the real-time pose of the EM sensor relative to a reference pose in the one-time calibration procedure. This “onsite” calibration can be done without additional hardware or having to provide an additional working channel in the endoscope. The calibration can be done in real-time during a surgical procedure, even if the endoscope is inside the patient.

It should be understood that the present invention will be described in terms of endoscopic procedures and endoscope devices; however, the teachings of the present invention are much broader and are applicable to any components that can be positioned within a patient for a medical procedure or the like, such as catheters, needles or other guided instruments. Embodiments described herein are initially located using a pre-operative imaging technique, e.g., CT scans, sonograms, X-rays, etc. Other techniques may also be employed.

It also should be understood that the present invention will be described in terms of medical instruments; however, the teachings of the present invention are much broader and are applicable to any instruments employed in tracking or analyzing complex biological or mechanical systems. In particular, the present principles are applicable to internal tracking procedures of biological systems, procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor”, “module” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD. The elements depicted in the FIGS. may be implemented in various combinations of hardware and provide functions which may be combined in a single element or multiple elements.

Referring now to the drawings in which like numerals represent the same or similar elements and initially to FIG. 1, a perspective view of a distal end portion 102 of an endoscope 100 is illustratively shown in accordance with one exemplary embodiment. The endoscope 100 in this embodiment includes an EM sensor 106 attached to the distal end portion 102 close to an aperture of a camera 108. Lights 109 are provided to illuminate internal areas for imaging. A long wire or cable 110 is employed to connect the sensor 106 to a tracking system 112, which can be either inside or outside the endoscope 100.

It is not ideal to keep the wire 110 outside the scope 100 since this complicates sterilization procedures, and may change the physician's feel of the scope 100 during the procedure. However, a standard endoscope has only one working channel 116 for inserting surgical devices such as forceps, catheters or brushes. The tracking wire 110 cannot permanently occupy the working channel 116. Therefore, a tracking sensor 118 is inserted through the working channel 116 during a procedure each time when tracking is needed. The tracking sensor 118 may employ the same tracking system 112 or a different tracking system. It is difficult or near impossible to keep a transformation between the camera 108 and the tracking sensor 118 unchanged every time the sensor 118 is inserted. Therefore, an onsite calibration system and method are provided. The tracking sensor 118 includes a locator feature 120, which may include a shape, indicia, 3D feature, etc. The locator feature 120 is employed to calibrate the camera 108 in real-time even if the scope 100 is inside a patient.

If the intrinsic parameters of the camera 108, e.g., image center, focal length, etc . . . ) and geometry of the image feature 120 are known, a transformation (e.g., in 6 degrees of freedom) between the image feature 120 and the camera 108 can be determined using a single image. For example, say the image feature 120 includes three vertices of a scalene triangle, assuming we know the physical distances between the vertices, the transformation between the camera 108 and the triangle (120) can be uniquely determined from one image of the triangle. This can be generalized to other feature types because any image feature 120 can be represented by a group of points.

Referring to FIG. 2, a system 200 for tracking an endoscope in accordance with the present principles is shown in accordance with one illustrative embodiment. System 200 preferably includes hardware and software components. System 200 includes a spatial tracking system 206 and a multiple degree-of-freedom (DOF) sensor 218 with an image identifiable feature(s) 220 (equivalent to features 120). The tracking system 206 and sensor 218 are preferably provided on an endoscope 100, which includes a camera 108. The tracking system 206 and sensor 218 may be part of an EM tracking system 232 which can monitor positions of devices in three-dimensional space.

A workstation (WS) or other processing device 222 including hardware configured to run software to acquire and display real-time medical procedure images on a display device 230. The workstation 222 spatially tracks a position and orientation of the sensor 218. The workstation 222 functions as a transformation module to provide the needed elements for transforming the position and orientation of features 220 in an image to preoperative images or models (real or virtual). Sensor 218 preferably includes a six DOF sensor; however fewer or greater numbers of degrees of freedom may be employed. The workstation 222 includes image processing software 224 in memory 225, which processes internal images including features 220 on the sensor 218. The software 224 computes the sensor's pose relative to the scope's camera 108.

The scope 100 includes a working channel 116. The sensor 218 is fed through the working channel 116 until the sensor 218 extends distally from the scope 100 and the feature or features 220 are visible in the camera 108. Since the scope includes tracking system 206, its position can be determined relative to the sensor 218. The visible feature(s) 220 permits the computation of the difference in position and yields a relative orientation between the system 206 and sensor 218. This computation can provide a transformation between the system 206/camera 108 and sensor 218, which can be employed throughout the medical procedure.

Processing device 222 may be connected to or be part of a computer system and includes memory 225 and an operating system 234 to provide the functionality as described in accordance with the present principles. Program 224 combines preoperative images (CT images) with real-time endoscope positions such that the preoperative images are rendered on a display 230 in real-time during the procedure.

The processing device or controller 222 includes a processor 238 that implements the program 224 and provides program options and applications. An input/output (I/O) device or interface 228 provides for real-time interaction with the controller 222, the endoscope 100 and sensor 218 to compare and show images. The interface 228 may include a keyboard, a mouse, a touch screen system, etc.

Referring to FIGS. 3A-3C, image-visible sensor features 220 are illustratively depicted. In one embodiment as depicted in FIG. 3A, features 220 may include a plurality of spaced circles 302. The circles 302 may be arranged in a repeating pattern or form a shape, such as a triangle or the like. The circles 302 may include a specific diameter or other known dimension (e.g., a distance between circles, etc.) or provide an angle or angles. The distances and/or angles may be employed to determine a position or orientation visually within an image. A known dimension may be compared within the image as a reference.

In another embodiment as shown in 3B, an arrow 304 may be employed for feature 220. The arrow may have a line segment of known length and the arrow may point in a direction relative to the camera 108 to assist in computing a pose of the scope (100).

In yet another embodiment as shown in 3C, a protrusion 306, divot 308 or other 3D feature may be formed on or in the sensor 218. This provides a three-dimensional feature for use in locating the sensor relative to the camera image. Other shapes, sizes, indicia and designs may also be employed.

Referring to FIG. 4, a transformation between the EM sensor and the bronchoscope's camera can be determined as follows. In block 402, a one-time calibration procedure may be conducted offline before a medical procedure, such as, e.g., a bronchoscopy. A multi-degree of freedom EM sensor is passed through the working channel of the scope until the features of the EM sensor can be identified in an image in block 404. In block 406, the sensor is then fixed relative to the bronchoscope's camera, which is referred to as a “reference pose”. The image of the bronchoscope is saved in block 408.

Referring to FIG. 5, an endoscopic image 450 is illustratively depicted of a sensor 218 having an image identifiable feature 220 thereon. The image is from a viewpoint of an endoscope camera.

Referring again to FIG. 4, in block 410, a transformation between the camera and the EM sensor is determined In block 411, this may include using a calibration phantom where a phantom image of the features is moved from a reference point and overlaid on the actual features depicted in the image. A difference is then computed for the movement of the calibration phantom.

In block 412, during a medical procedure, the scope is tracked. In block 413, the EM sensor is passed through the working channel of the bronchoscope until the features of the EM sensor can be identified in the camera image. The image is processed to determine the real-time pose of the EM sensor relative to the reference pose in the one-time calibration procedure (off-line calibration) in block 414. The real-time transformation between the EM sensor and the camera can be computed as the following:

T_{EMsensor RealtimePose}^{Camera}=T_{EMsensor ReferencePose}^{Camera}·T_{EMsensor RealtimePose}^{EMsensor ReferencePose} (1)

where T_{A}^{B} is the transformation from B to A. Therefore, T_{EMsensor ReferencePose}^{Camera} is the calibration result of block 402 and T_{EMsensor RealtimePose}^{EMsensor ReferencePose} is relative transformation between the pose 1 and pose 2 of the EM sensor. In block 415, an endoscopic image may be registered to a preoperative image (e.g., a CT image).

The scope is placed under the guidance of EM tracking in block 415. The EM sensor can be pulled out of the bronchoscope's working channel in block 416. The medical procedure then continues, and surgical devices can be inserted into the working channel to take biopsy samples or perform other actions, in block 418.

In interpreting the appended claims, it should be understood that:

a) the word “comprising” does not exclude the presence of other elements or acts than those listed in a given claim;

b) the word “a” or “an” preceding an element does not exclude the presence of a plurality of such elements;

c) any reference signs in the claims do not limit their scope;

d) several “means” may be represented by the same item or hardware or software implemented structure or function; and

e) no specific sequence of acts is intended to be required unless specifically indicated.

Having described preferred embodiments for systems and methods for real-time endoscope calibration (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A sensor tracking device, comprising
a sensor (118) configured on a wire (110) and adapted to fit in a working channel (116) of an endoscope (100); and
at least one image identifiable feature (120) formed on a distal end portion of the sensor which is identifiable when the sensor is extended from the endoscope such that an image of the at least one image identifiable feature collected by the endoscope permits a determination of a pose of the endoscope.

2. The sensor tracking device as recited in claim 1, wherein the sensor (118) includes a multiple degree of freedom sensor.

3. The sensor tracking device as recited in claim 1, wherein the endoscope (100) includes an imaging device (108) configured to collect images from a distal end portion of the endoscope.

4. The sensor tracking device as recited in claim 1, wherein the at least one image identifiable feature (120) includes a reference dimension.

5. The sensor tracking device as recited in claim 1, wherein the at least one image identifiable feature (120) includes a reference angle.

6. The sensor tracking device as recited in claim 1, wherein the at least one image identifiable feature (120) includes one or more shapes which indicate an orientation of the sensor.

7. The sensor tracking device as recited in claim 1, wherein the at least one image identifiable feature (120) includes an integrally formed shape on the sensor.

8. A system for tracking an endoscope, comprising:
an endoscope (100) having a working channel (116), a spatial tracking system (206) and a distally disposed imaging device (108);
a sensor (218) configured on a wire and adapted to fit in the working channel;
at least one image identifiable feature (220) formed on a distal end portion of the sensor which is identifiable when the sensor is extended from the endoscope; and
a transformation module (222) configured to compute a pose of the endoscope by employing a position of an image of the at least one image identifiable feature collected by the imaging device and a position of the spatial tracking system.

9. The system as recited in claim 8, wherein the sensor (218) includes a multiple degree of freedom sensor.

10. The system as recited in claim 8, wherein the at least one image identifiable feature (220) includes a reference dimension.

11. The system as recited in claim 8, wherein the at least one image identifiable feature (220) includes a reference angle.

12. The system as recited in claim 8, wherein the at least one image identifiable feature (220) includes one or more shapes which indicate an orientation and a position of the sensor.

13. The system as recited in claim 8, wherein the at least one image identifiable feature (220) includes an integrally formed shape on the sensor.

14. The system as recited in claim 8, wherein the transformation module includes an image processor (224) configured to identify the at least one image identifiable feature and compute a position and orientation of the sensor relative to the imaging device.

15. A method for tracking an endoscope, comprising:
calibrating (402) a transformation between a distally disposed imaging device of an endoscope and a sensor having at least one image identifiable feature formed on a distal end portion of the sensor which is identifiable when the sensor is extended from the endoscope; and
tracking (412) the endoscope by:
passing (413) the sensor through a working channel of the endoscope until the at least one image identifiable feature is imaged; and
computing (414) a current pose of the endoscope using an image of the at least one image identifiable feature and the transformation.

16. The method as recited in claim 15, wherein calibrating (402) includes employing a calibration phantom image (411).

17. The method as recited in claim 15, wherein the at least one image identifiable feature (120) includes at least one of a reference dimension and a reference angle.

18. The method as recited in claim 15, wherein the at least one image identifiable feature (120) includes one or more shapes which indicate an orientation and position of the sensor.

19. The method as recited in claim 15, wherein tracking (412) the endoscope is performed in a patient during a procedure.

20. The method as recited in claim 15, further comprising removing (416) the sensor from the working channel when the endoscope has been registered with a tracking system.
